(19) United States
(12) Patent Application Publication
(10) Pub. No.: US 2016/0015403 A1
(43) Pub. Date: Jan. 21, 2016

(54) MEDICAL RETRIEVAL DEVICES AND METHODS

(71) Applicant: Boston Scientific Scimed, Inc., Maple Grove, MN (US)

(72) Inventors: Man Minh NGUYEN, Harvard, MA (US); John Allen HINGSTON, Framingham, MA (US); Paul SMITH, Smithfield, RI (US)

(21) Appl. No.: 14/798,843

(22) Filed: Jul. 14, 2015

Related U.S. Application Data
(60) Provisional application No. 62/024,588, filed on Jul. 15, 2014.

(51) Int. Cl. A61B 17/221; A61B 17/50

(52) U.S. Cl. CPC A61B 17/221; A61B 17/50; A61B 2017/2212; A61B 2017/2215; A61B 2017/2217

(57) ABSTRACT

A medical device may include a basket having a proximal end and a distal end. The medical device may further include a longitudinally extending member extending coupled to the proximal end of the basket. The medical device may further include a closure member extending along the longitudinally extending member and circumferentially about the distal end of the basket. Manipulation of the closure member may be configured to transition the distal end of the basket between a first closed position and a second open position.

### CROSS REFERENCE TO RELATED APPLICATIONS

This application claims the benefit of U.S. Provisional Application No. 62/024,588, filed Jul. 15, 2014, the disclosure of which is incorporated herein by reference in its entirety.

### TECHNICAL FIELD

The disclosure generally relates to medical retrieval devices such as, for example, baskets, for retrieving material in a body of a patient. More particularly, the disclosure generally relates to medical retrieval baskets with release mechanisms and methods of use.

### BACKGROUND

Medical retrieval devices are often utilized for removing organic material (e.g., blood clots, tissue, and biological concretions such as urinary, biliary, and pancreatic stones) and inorganic material (e.g., components of a medical device or other foreign matter), which may obstruct or otherwise be present within a patient's body cavities. For example, concretions can develop in certain parts of the body, such as in the kidneys, pancreas, ureter, and gallbladder. Minimally invasive medical procedures are used to remove these concretions through natural orifices, or through an incision, such as during a percutaneous nephrolithotomy (PNCL) procedure. Other procedures may include endoscopic retrograde cholangiopancreatography (ERCP) which is a procedure for treating the bile and pancreatic ducts of a patient. Further, lithotripsy and ureteroscopy, for example, are used to treat urinary calculi (e.g., kidney stones) in the ureter of a patient.

One problem commonly associated with medical retrieval baskets occurs where the stone or other material is too large to be removed intact from the body tract after it has been captured within the medical retrieval basket. In such cases, medical professionals may attempt to dislodge the stone or other material from the basket. If the stone or material cannot be removed from the basket in this manner, however, a medical professional often must use an emergency rescue handle to hopefully break the stone in the basket. If the stone or material doesn't break, the basket tends to break along its drive wire. The patient must then undergo surgery to remove the broken basket.

### SUMMARY

Examples of the present disclosure relate to, among other things, medical retrieval devices and related methods of use. Each of the examples disclosed herein may include one or more of the features described in connection with any of the other disclosed examples.

In one example, a medical device includes a basket having a proximal end and a distal end. The medical device further includes a longitudinally extending member coupled to the proximal end of the basket. The medical device also includes a closure member extending through the longitudinally extending member and circumferentially about the distal end of the basket. Manipulation of the closure member is configured to transition the distal end of the basket between a first closed position and a second open position.

Examples of the medical device may include one or more of the following features: the longitudinally extending member may include at least one of a coil or a hypotube; the closure member may include at least one of a string, a suture, or a wire; a handle may be coupled to a sheath, wherein the handle may include an actuator configured to reciprocally move the basket from a position within the sheath to a position extending distally of the sheath; the actuator may include at least one of a sliding mechanism, a rotating mechanism, or a pushing mechanism; a closure member actuator may be configured to reciprocally move the closure member relative to the longitudinally extending member, thereby moving the basket between the first closed position and the second open position; the basket may be fixed relative to the longitudinally extending member; the basket may be movable relative to the longitudinally extending member; the longitudinally extending member may include at least one stop configured to limit the axial movement of the basket relative to the longitudinally extending member; the closure member may include a first portion extending within the longitudinally extending member towards the distal end of the basket, a second portion encircling the distal end of the basket, and a third portion extending within the longitudinally extending member towards the proximal end of the basket; the proximal end of the basket may define an opening to the basket larger than openings at the distal end of the basket; wherein the proximal end of the basket includes only two longitudinally extending legs and the distal end of the basket comprises a net of more than two legs; the longitudinally extending member may extend along a lateral side of basket; the closure member may be interlaced through legs at the distal end of the basket; and the closure member may comprise a purse-string suture at the distal end of the basket.

In another example, a medical device comprises a sheath and a stent. The stent has a proximal end, a distal end, and defines a lumen extending therethrough. The stent is configured to transition between a first collapsed position within the sheath and a second expanded configuration extending distally of the sheath. A selectively expandable member is positioned at a distal end of the stent and expandable independent of the stent. The expandable member is configured to reduce a diameter of the lumen when expanded.

Examples of the medical device may include one or more of the following features: the stent may be include a coating that at least partially extends about the expandable member; the stent may be fixedly connected to a handle via a member extending through the sheath; and the expandable member may have a toroidal shape and may comprise an inflatable pliant balloon.

In another example, a method comprises delivering a basket to an area of interest within a patient. The basket is configured to transition between a first retracted configuration within a sheath and a second deployed configuration extending distally of the sheath. The basket includes a longitudinally extending member coupled to a proximal end of the basket, and a closure member extending along the longitudinally extending member and circumferentially about a distal end of the basket. Manipulation of the closure member is configured to transition the distal end of the basket between a first closed position and a second open position. The method further includes moving the basket to the deployed configuration and capturing an object of interest within the basket. Additionally, the method includes extending the closure member relative to the longitudinally extending member to open the distal end of the basket.

Examples of the medical device may include one or more of the following features: releasing the object of interest; the closure member may include a first portion extending within the longitudinally extending member towards the distal end of the basket, a second portion encircling the distal end of the basket, and a third portion extending within the longitudinally extending member towards the proximal end of the basket; retracting the closure member relative to the longitudinally extending member to close the distal end of the basket; and moving the basket relative to the longitudinally extending member.

It may be understood that both the foregoing general description and the following detailed description are exemplary and explanatory only and are not restrictive of the disclosure, as claimed.

### BRIEF DESCRIPTION OF THE DRAWINGS

The accompanying drawings, which are incorporated in and constitute a part of this specification, illustrate exemplary aspects of the present disclosure and together with the description, serve to explain the principles of the disclosure.

FIG. 1 illustrates an exemplary handle of a medical device according to an aspect of the disclosure;

FIG. 2 illustrates an exemplary basket of an exemplary medical device according the an aspect of the present disclosure, in a closed configuration;

FIG. 3 illustrates the basket of FIG. 2 in an open configuration;

FIG. 4 illustrates a further exemplary basket of an aspect of the present disclosure;

FIG. 5 illustrates a still further exemplary basket of an aspect of the present disclosure;

FIG. 6 illustrates a partially-deployed stent according to another aspect, in an open configuration;

FIG. 7 illustrates the partially deployed stent of FIG. 6 in a closed configuration; and

FIG. 8 illustrates a partially-deployed stent according to yet another aspect of the present disclosure, in a closed configuration.

### DETAILED DESCRIPTION

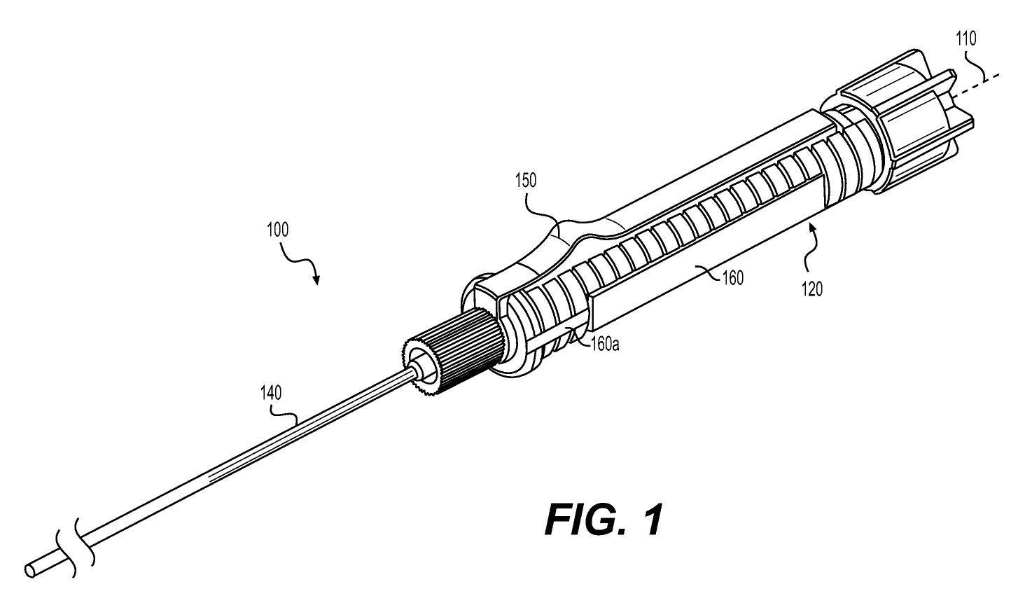

As shown in FIG. 1, a medical device 100 according to an exemplary aspect of the present disclosure may include a handle 120 extending along a central longitudinal axis 110. Handle 120 may be configured to selectively extend and retract a medical retrieval device (e.g., a basket) 130 (not shown in FIG. 1) relative to a sheath 140, as will be described in further detail below. Accordingly, handle 120 may include an actuator 150 configured to reciprocally move basket 130 relative to sheath 140.

Actuator 150 may include, for example, a sliding mechanism, a rotating mechanism, a pushing mechanism, or the like. In some examples, the actuator 150 may be coupled to a longitudinally extending hollow member 180 (not shown in FIG. 1) in any conventional manner to control the movement of the basket 130. The hollow member 180 may be a coil or hypotube or other suitable drive member having sufficient columnar strength to advance and retract basket 130 relative to sheath 140. For example, upon actuation of actuator 150 in a first direction (e.g., distal direction), the hollow member 180 may be configured to move the basket 130 between a collapsed position within sheath 140 and an expanded position distal of the sheath 140, as will be described in further detail below.

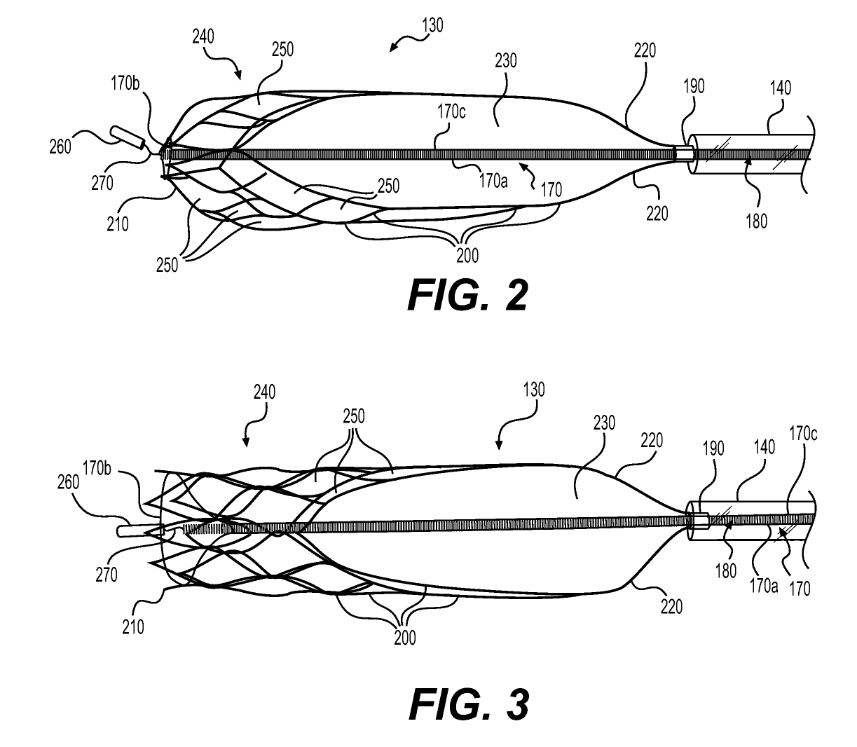

Handle 120 may include an additional actuator 160 on a side of handle 120. Actuator 160 may be configured to reciprocally move a closure member 170 (e.g., a string, suture, and/or wire) (see FIG. 2) relative to hollow member 180 so as to open and close a distal end of basket 130, as will be described in further detail below. For example, upon actuation of actuator 160 in a first direction (e.g., distal direction) along a slot 160a, closure member 170 may move relative to hollow member 180 thereby providing slack in the closure member 170 and allowing the distal end of basket 130 to open (FIG. 3), and vice versa. It is understood that the closure member 170 may comprise a material and/or construction having sufficient columnar strength to advance and retract relative to hollow member 180. It is understood that while actuator 160 has been described as a sliding mechanism, additional or alterative mechanisms may be used such as, for example, a rotating mechanism, a pushing mechanism, or the like. It should be noted that in some examples, closure member 170 may extend alongside an external surface of member 180 and in such examples, member 180 need not be hollow. Rather, member 180 may be any sufficient rod, wire, or the like.

Sheath 140 may include any appropriate polyimide tube or a hollow member of another material that exhibits suitable radial flexibility, axial stiffness, and biocompatibility. In addition, sheath 140 may have any appropriate outer dimension (e.g., diameter) and cross-sectional shape configured for insertion through a channel of a conventional insertion device such as, for example, a scope device (such as a ureteroscope, endoscope, colonoscope, etc.), a catheter, a sheath, or the like.

As shown in FIG. 2, basket 130 may include a proximal collar 190, and a distal end 210, and may comprise a plurality of interconnected legs or struts (e.g., wires) 200. Basket 130 may have any number or pattern of legs 200. The number or pattern of legs 200 may be selected on the basis of a number of parameters such as, for example, the size of the body lumen/cavity into which the basket 130 is inserted, the size of the stone or other material to be removed, the consistency of the stone or other material, the position of the stone or other material, and the location and/or orientation of the stone or other material within a patient.

In the aspect shown in FIG. 2, for example, the legs 200 of basket 130 may include two longitudinally extending proximal legs 220 coupled to or monolithically formed with a collar 190. Proximal legs 220 may define an opening or entrance zone 230 to the basket 130. That is, proximal legs 220 may be spaced on opposite lateral sides of basket 130 such that zone 230 is configured to receive a stone or other object therein. In use, as will be described in further detail below, a medical professional may move, arrange, or otherwise manipulate basket 130 such that a stone or other material may be received within (e.g., pass into) the basket 130 through the zone 230 defined by proximal legs 220.

A plurality of interconnected legs 200 comprising a distal net 240 may extend distally of proximal legs 220. As shown in FIG. 2, a density of legs 200 in net 240 is greater than a density of proximal legs 220. Net 240 may form a number of small apertures 250 which may be sized to permit fluid (e.g., blood) to flow through, but which are small enough to prevent a stone or other material from exiting basket 130. In other words, once a stone or other material is received within basket 130 (e.g., via zone 230), it is trapped therein by net 240. The size of apertures 250 may be selected such that they are large enough to permit fluid (e.g., blood) flow while preventing the release of a stone therethrough. Additionally, apertures 250 may have any appropriate shape. For example, apertures 250 may be almond shaped as shown in FIGS. 2 and 3. Alternatively, apertures 250 may be circular shaped, oval shaped, polygonal shaped, and/or diamond shaped, and/or the like. Additionally, in some examples, the size and/or shape of apertures 250 may vary along the length of net 240. That is, apertures 250 proximate to proximal legs 220 may be shaped and/or sized differently than apertures nearer to distal end 210 of basket 130, and vice versa.

Collar 190 may be configured to couple, secure, or otherwise attach basket 130 to hollow member 180. As noted above, in some examples, collar 190 may be coupled to legs 220 via any appropriate method including, for example, welding, adhesives, and/or tying. In other examples, however, collar 190 may be monolithically formed with basket 130 such that basket 130 and collar 190 form a one-piece construction. Further, collar 190 may be coupled to the hollow member 180 through any appropriate means such as, for example, welding and/or adhesives.

In accordance with aspects of the present disclosure, a method of basket 130 formation involves starting with a hollow tube or cannula. For axial basket features (e.g., features extending substantially axially along a longitudinal axis of the underlying basket 130 structure) slots are cut lengthwise along the exterior of the cannula wall substantially parallel to the longitudinal axis of the cannula. For helical, spiral, or other features that are not solely axial in direction (e.g., features that extend at an angle with respect to a longitudinal axis of the tube), diagonal slots are cut with both an axial component and a radial component. After cutting and cleaning, the resulting tubes are compressed lengthwise (e.g., expanded radially) to spread the legs 200 and manipulate the legs 200 into a desired shape. This shape is then annealed into the material to form a desired basket configuration. The slotting pattern can be formed according to a laser cutting process. Other machining processes are possible, such as chemical etching, EDM (Electrical Discharge Machining), and joining individual legs 200 together by welding or other attachment methods.

The basket 130 configuration according to examples of the present disclosure can be made at least partially of a shape-memory material. Shape-memory material is a material that can be formed into a particular shape, retain that shape during resting conditions (e.g., when the shaped material is not subject to external forces or when external forces applied to the shaped material are insufficient to substantially deform the shape), be deformed into a second shape when subjected to a sufficiently strong external force, and revert substantially back to the initial shape when external forces are no longer applied. Examples of shape memory materials include synthetic plastics, stainless steel, and superelastic metallic alloys of nickel/titanium (e.g., Nitinol), copper, cobalt, vanadium, chromium, iron, or the like. Alternative basket materials include, but are not limited to, other metal alloys, powdered metals, ceramics, thermal plastic composites, ceramic composites, and polymers. Also, combinations of these and other materials can be used.

Closure member 170 may include a first end coupled to actuator 160 and a second end, fixedly anchored to handle 120. Additionally, closure member 170 may include a length extending within the hollow member 180 from the first end coupled to the actuator 160 distally toward distal end 210 of basket 130, exiting hollow member 180 and encircling the distal end 210 of basket 130, and reentering the hollow member 180 and extending proximally to the second end of closure member 170 fixedly anchored to handle 120. As such, closure member 170 may be folded, bent, doubled over, or otherwise arranged such that a first portion 170a of closure member 170 extends distally toward distal end 210, a second portion 170b encircles distal end 210, and a third portion 170c extends proximally toward handle 120. Second portion 170b of closure member 170 may be weaved and/or intertwined through apertures 250 on the distal end 210 of basket 130. For example, the second portion 170b of closure member 170 may be passed through apertures 250 in a manner similar to a purse-string suture such that in the arrangement shown in FIG. 2, the distal end 210 of basket 130 is tightly or snugly closed or cinched shut. First and third portions 170a and 170c may exit the hollow member 180 through any appropriate means. For example, each of 170a and 170c may exit the hollow member through openings, holes, or other apertures in a side wall of hollow members, and through the distal end of the hollow member 180.

In some examples, hollow member 180 may include a single lumen through which each of the first 170a and third 170c portions of closure member 170 extend. In additional and/or alternative examples, however, hollow member 180 may include a plurality of lumens such that the first portion 170a of closure member 170 extends through a first lumen of hollow member 180, while the third portion 170c of closure member 170 extends through a second lumen of hollow member 180. In some examples, the lumen(s) of hollow member 180 and/or the closure member 170 (or some portion 170a-c thereof) may comprise a lubricious coating configured to reduce friction and ease movement of closure member 170 relative to hollow member 180.

A distal end of hollow member 180 may be coupled to an atraumatic tip 260 via a coupler 270 such as, for example, a wire or other extension member. Tip 260 may prevent inadvertent damage to tissue or surrounding body structures which may be caused by movement of basket 130 within a body lumen or cavity of a patient. In some examples, tip 260 may comprise a cap with a rounded end.

Referring to FIGS. 1-3, a method of operating medical device 100 will be described. First, a medical professional may introduce medical device 100 into the body of a patient through any appropriate means such as, for example, a scope or similar introducer device. Once positioned, a medical professional may manipulate actuator 150 such that basket 130 extends distally of sheath 140 as shown in FIG. 2. Once basket 130 extends distally of the sheath 140, the basket 130 may expand to form a cage configured to capture and retrieve a stone or other material from within the body of the patient.

During use, a stone or other material may be captured within the basket 130 through zone 230. For example, a medical professional may position proximal legs 220 of basket 130 adjacent a stone or other material and urge the stone into the basket 130 through zone 230. In order to remove the stone, the medical professional may pull or otherwise proximally retract medical device 100 such that the stone or other material is trapped within the closed distal end 210 of basket, defined by net 240, and may be removed from the body of the patient. Additionally or alternatively, the medical professional can partially withdraw basket 130 into sheath 140 to tightly enclose the stone or other object in the partially withdrawn basket.

If, however, the stone or material is too large to be removed intact from the body after it has been captured within basket 130, a medical professional may loosen closure member 170 to release the oversized stone or material from the basket 130 via actuator 160. For example, a medical professional may urge actuator 160 in a distal direction along slot 160a, thereby advancing first portion 170a of closure member 170 distally and consequently causing second portion 170b of closure member 170 to expand and/or loosen around distal end 210 of basket 130. Once loosened, distal end 210 of basket 130 may open, as shown in FIG. 3, such that an oversized stone or material may be released from basket 130.

Once released, a medical professional may retract actuator 160 in a proximal direction such that basket 130 may return to its closed configuration as shown in FIG. 2. For example, movement of actuator 160 in a proximal direction may retract first portion 170a of closure member proximally, and consequently, causes second portion 170b of closure member 170 to tighten or cinch shut the distal end 210 of basket 130. Accordingly, a medical professional may redirect the medical device 100 to capture a different stone and/or material within the body of a patient.

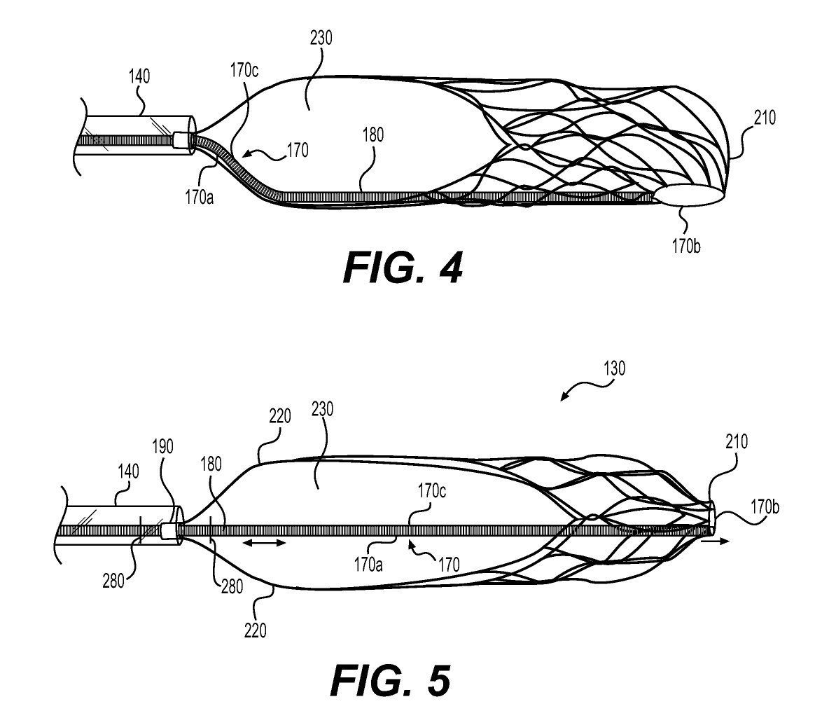

In an additional example, as shown in FIG. 4, hollow member 180 may extend along a lateral side of basket 130 such that hollow member 180 is removed from and/or enlarges zone 230. In such an example, loosening and tightening closure member 170 via actuator 160 may create a hood-type arrangement as shown. That is, the distal end 210 of basket 130, when cinched shut via actuator 160, forms an enclosure offset from a central longitudinal axis of the basket 130. In some examples, hollow member 180 may have be comprised of shape-memory material such that once extended from sheath 140, hollow member 180 may acquire the configuration shown in FIG. 4. Beyond the positioning of hollow member 180 along a lateral side of basket 130, the example shown in FIG. 4 may function in a manner similar to the examples shown in FIGS. 2 and 3.

In an additional example, as shown in FIG. 5, hollow member 180 may be reciprocally moved relative to basket 130 so as to stretch basket 130 until the distal end 210 cinches shut or collapses in on itself. In such an example, actuator 150 may be a multi-step actuator in which advancement of actuator 150 a first distance in the distal direction will cause basket 130 to extend distally of sheath 140. In addition, further advancement of actuator 150 in the distal direction may stretch basket 130 such that the distal end 210 of basket 130 cinches closed. In such examples, actuator 150 may provide a tactile response for the medical professional. For example, in some aspects, upon advancement of actuator 150 the first distance, a medical professional may hear and/or feel a click, noise, or other movement indicating that the basket 130 is distal of the sheath 140. Additionally, upon further advancement of actuator 150 in the distal direction, a medical professional may hear and/or feel a click, noise, or other movement indicating that the basket 130 has been stretched and the distal end 210 is cinched shut.

Contrary to the examples described with respect to FIGS. 2-4, in the example of FIG. 5, collar 190 may be configured to move relative to hollow member 180, thus permitting the basket 130 to be stretched. Additionally, hollow member 180 may include one or more stops 280 limiting movement of collar 190, and therefore basket 130, relative to hollow member 180. Stops 280 may be coupled to or monolithically formed with hollow member 180 and may comprise any appropriate construction to halt movement of collar 190 relative to hollow member 180 beyond a predefined range of motion. For example, stops 280 may comprise any one or more or a combination of discs, protrusions, and the like extending radially outward from an outer surface of hollow member 180.

In use, a medical professional may introduce medical device 100 into the body of a patient through any appropriate means such as, for example, a scope or similar introducer device. Once positioned, a medical professional may urge actuator 150 a first distance in the distal direction such that basket 130 extends distally of sheath 140. Once basket 130 extends distally of the sheath 140, the actuator 150 may be further advanced in the distal direction such that a distal end 210 of basket 130 may collapse upon itself to thereby form a cage configured to capture and retrieve a stone or other material from within the body of the patient as shown in FIG. 5.

During use, a stone or other material may be captured within the basket 130 through zone 230. For example, a medical professional may position proximal legs 220 of basket 130 adjacent a stone or other material and urge the stone into the basket 130 through zone 230. In order to remove the stone, the medical professional may pull or otherwise proximally retract medical device 100 such that the stone or other material is trapped within the closed distal end 210 of basket, defined by net 240, and may be removed from the body of the patient. If, however, the stone or material is too large to be removed intact from the body after it has been captured within basket 130, a medical professional may retract actuator 150 proximally such that the distal end 210 is no longer stretched to the point of cinching shut, and therefore, an oversized stone or object may be released through an open distal end 210 of basket 130 (FIG. 3).

While some exemplary methods include delivery of the medical device 100 in the closed position (as shown in FIG. 2) and as described above, in some examples, medical device 100 may be introduced into the body of a patient in the open configuration as shown in FIG. 3. In such an example, a medical professional may distally advance basket 130 such that a stone or other object may be received into the basket 130 via open distal end 210 of basket 130. Once received within the basket 130, a medical professional may close the distal end 210 of the basket 130 via actuator 160 such that the stone or other material may be trapped therein. In order to remove the stone, the medical professional may pull or otherwise proximally retract medical device 100 such that the stone or other material is trapped within the closed distal end 210 of basket, defined by net 240, and may be removed from the body of the patient. In another alternative method, medical device 100 may be introduced into the body of a patient in the closed configuration as shown in FIG. 2, subsequently opened as shown in FIG. 3, and then moved to receive a stone or other object therein. Once received within the basket 130, a medical professional may close the distal end 210 of the basket 130 via actuator 160 such that the stone or other material may be trapped therein. In order to remove the stone, the medical professional may pull or otherwise proximally retract medical device 100 such that the stone or other material is trapped within the closed distal end 210 of basket, defined by net 240, and may be removed from the body of the patient.

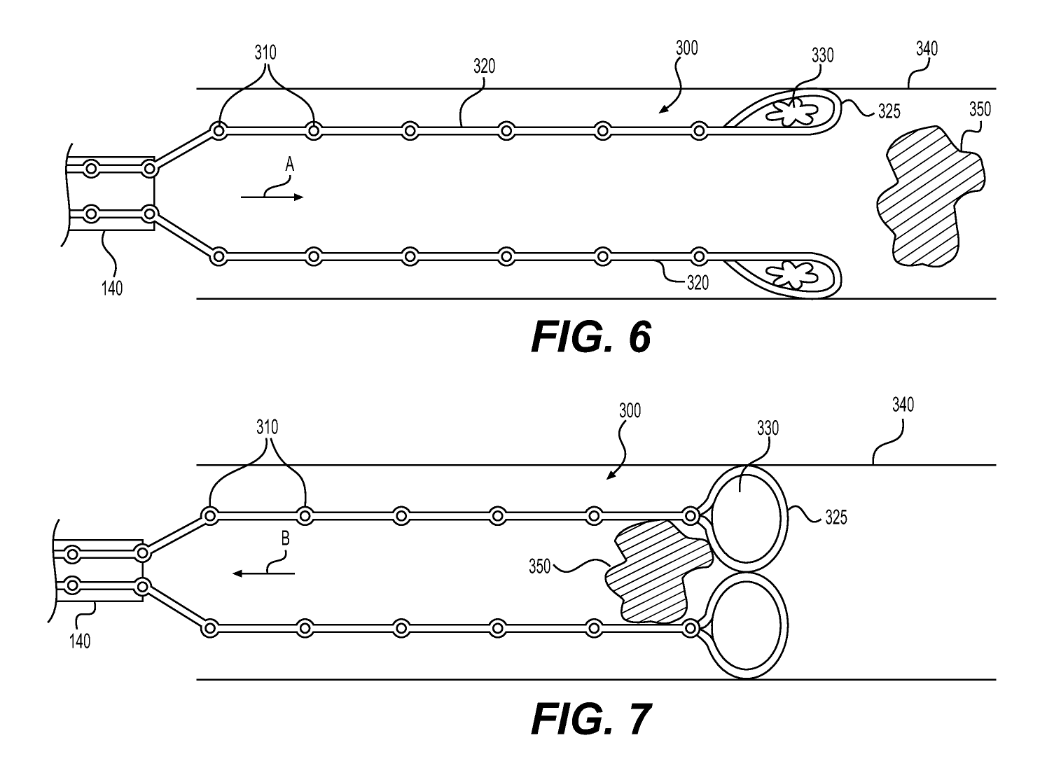

In a further example, as shown in FIGS. 6 and 7, a medical device 300 may include a self-expanding basket and/or stent 310. While referred to herein as a stent, it should be understood that stent 310 may be used or described as basket without departing from the scope of this disclosure. Stent 310 may be configured to transition between a collapsed configuration within a sheath 140, and a partially expanded configuration as shown in FIGS. 6 and 7. In such examples, actuator 150 may be coupled to a proximal end of sheath 140 and configured to reciprocally move sheath 140 relative to stent 310. For example, proximally retracting actuator 150 relative to handle 120 may proximally withdraw sheath 140 relative to stent 310 thereby allowing any portion of stent 310 extending distally of sheath 140 to radially expand, and vice versa.

Stent 310 may include any appropriate self-expanding mesh or coil structure. For example, stent 310 may include a braided or twisted lattice of wire(s), a helical or semi-helical spiral, and/or a plurality of undulating, corrugated, or sinusoidal rings. Additionally, stent 310, according to examples of the present disclosure, can be made at least partially of a shape-memory material such as, for example, Elgiloy, synthetic plastics, stainless steel, and superelastic metallic alloys of nickel/titanium (e.g., Nitinol), copper, cobalt, vanadium, chromium, iron, or the like. Alternative stent 310 materials include, but are not limited to, other metal alloys, powdered metals, ceramics, thermal plastic composites, ceramic composites, and polymers. Also, combinations of these and other materials can be used.

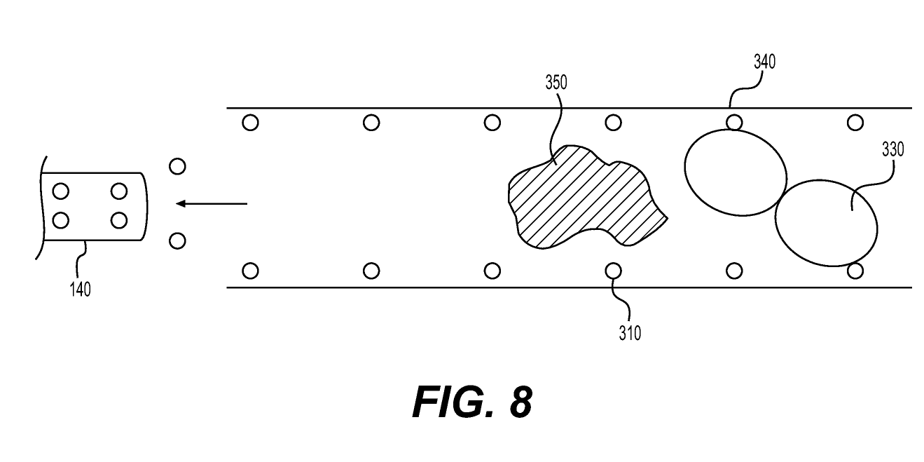

In some examples, as shown in FIGS. 6 and 7, stent 310 may include a thin coating or covering 320 extending along the stent 310. In other examples, stent 310 may be free from any coating 320, as shown in FIG. 8. Coating 320 may be configured to prevent stent 310 from causing unintentional damage to surrounding tissue and/or prevent damage to the stent 310 from a stone or other material entrapped by the stent 310. Coating 320 may be comprised of any suitable biocompatible polymer such as, for example, silicon. In some examples, stent 310 and/or coating 320 may include a lubricious coating configured to reduce friction and ease movement of stent 310 and/or coating 320 relative to sheath 140, as will be described in further detail below.

An inflatable member such as a pliant balloon 330 may be coupled to a distal end of stent 310. In some examples, balloon 330 may be encased within coating 320 as shown in FIGS. 6 and 7. For example, coating 320 may include an extension 325 surrounding and maintaining balloon 330 adjacent the distal end of stent 310. In other examples which do not include coating 320, as shown in FIG. 8, balloon 330 may be directly coupled to stent 310 via any appropriate mechanism such as, for example, adhesives. Balloon 330 may be comprised of any appropriate pliable thin gauge plastic or other biocompatible material which is capable of stretching to only a minor degree (or no degree) to provide a controlled maximum diameter when fully inflated. Balloon 330 may be inflated via an inflation lumen (not shown). The inflation lumen may extend along the length of the stent 310 to the balloon 330. The inflation lumen may have a small diameter such as, about 6 French (2 mm), similar to an angioplasty balloon. In such examples actuator 160 may be fluidly coupled to the inflation lumen which is in in turn, in fluid communication with the balloon 330. For example, distally advancing actuator 160 along slot 160a relative to handle 120 may activate a pump and/or open a valve within the inflation lumen and cause balloon 330 to inflate, and vice versa. Balloon 330 may be inflated with any appropriate medium such as, saline, air, gas, or other fluid.

In some examples, balloon 330 may be toroidal (e.g., doughnut) shaped. In the aspects shown in FIGS. 6 and 7, for example, the balloon 330 may extend across the distal end of stent 310 perpendicular to a longitudinal axis of the stent 310. In the example shown in FIG. 8, however, the balloon 330 may be angled such that the balloon 330 may extend across the distal end of stent 310 in a non-perpendicular fashion to the longitudinal axis of the stent 310. In such examples, an outer diameter of the stent 310 may be reduced relative to the examples of FIGS. 6 and 7, when using the same diameter balloon 330.

In use, a medical professional may introduce medical device 300 into the body of a patient through any appropriate means such as, for example, a scope or similar introducer device. For example, the medical professional may deliver medical device 300 into a body lumen 340 of a patient. Once positioned, a medical professional may manipulate actuator 150 such that sheath 140 is retracted relative to stent 310. Once stent 310 extends distally of the sheath 140, the stent 310 may radially expand as shown in FIGS. 6-8.

Once expanded, the medical professional may urge medical device 300 distally along lumen 340 in direction A as shown in FIG. 6. Upon reaching a stone 350, medical device 300 may be further urged distally of stone 350 and the medical professional may actuate actuator 160 so as to inflate balloon 330 as shown in FIG. 7. Inflation of balloon 330 closes the distal end or narrows a diameter of stent 310 such that the stone 330 is preventing from exiting stent 310. Accordingly, once the medical professional inflates the balloon 330, he or she may then retract balloon 300 in direction B as shown in FIG. 7. Accordingly, a medical professional may retrieve the stone 350 from the body lumen 340 of the patient.

If, however, the stone 350 is too large to be removed intact from the body after it has been captured within stent 310, a medical professional may allow the balloon 330 to deflate via actuator 160. For example, a medical professional may urge actuator 160 in a proximal direction along slot 160a, thereby deflating balloon 330. Once deflated, the distal end of stent 310 may open or the diameter may be widened such that an oversized stone 350 may be released from stent 310, as shown in FIG. 6.

Once released, a medical professional may distally redirect the medical device 300 to capture a different stone and/or material within the body of a patient, reinflate the balloon 330 to close the distal end or narrow a diameter of stent 310 such that the stone 330 is preventing from exiting stent 310. Accordingly, once the medical professional inflates the balloon 330, he or she may then retract balloon 300 in direction B as shown in FIG. 7. Accordingly, a medical professional may retrieve the stone 350 from the body lumen 340 of the patient.

While principles of the present disclosure are described herein with reference to illustrative aspects for particular applications, it should be understood that the disclosure is not limited thereto. Those having ordinary skill in the art and access to the teachings provided herein will recognize additional modifications, applications, examples, and substitution of equivalents all fall within the scope of the aspects and examples described herein. Accordingly, the disclosure is not to be considered as limited by the foregoing description.

We claim:

1. A medical device, comprising:
a basket having a proximal end and a distal end;
a longitudinally extending member coupled to the proximal end of the basket; and
a closure member extending through the longitudinally extending member and circumferentially about the distal end of the basket, wherein manipulation of the closure member is configured to transition the distal end of the basket between a first closed position and a second open position.

2. The medical device of claim 1, wherein the longitudinally extending member includes at least one of a coil or a hypotube.

3. The medical device of claim 1, wherein the longitudinally extending member includes at least one of a string, a suture, or a wire.

4. The medical device of claim 1, further comprising:
a handle coupled to a sheath, wherein the handle includes an actuator configured to reciprocally move the basket from a position within the sheath to a position extending distally of the sheath.

5. The medical device of claim 4, wherein the actuator includes at least one of a sliding mechanism, a rotating mechanism, or a pushing mechanism.

6. The medical device of claim 1, further comprising:
a closure member actuator configured to reciprocally move the closure member relative to the longitudinally extending member, thereby moving the basket between the first closed position and the second open position.

7. The medical device of claim 1, wherein the basket is fixed relative to the longitudinally extending member.

8. The medical device of claim 1, wherein the basket is movable relative to the longitudinally extending member.

9. The medical device of claim 8, wherein the longitudinally extending member includes at least one stop configured to limit the axial movement of the basket relative to the longitudinally extending member.

10. The medical device of claim 1, wherein the closure member includes a first portion extending within the longitudinally extending member towards the distal end of the basket, a second portion encircling the distal end of the basket, and a third portion extending within the longitudinally extending member towards the proximal end of the basket.

11. The medical device of claim 1, wherein the longitudinally extending member extends along a lateral side of the basket.

12. A medical device, comprising:
a sheath;
a stent having a proximal end, a distal end, and defining a lumen extending therethrough, the stent configured to transition between a first collapsed position within the sheath and a second expanded configuration extending distally of the sheath; and
a selectively expandable member positioned at a distal end of the stent, wherein the expandable member is expandable independently of the stent and configured to reduce a diameter of the lumen when expanded.

13. The medical device of claim 12, wherein the stent includes a coating that at least partially extends about the expandable member.

14. The medical device of claim 12, wherein the stent is fixedly connected to a handle via a member extending through the sheath.

15. The medical device of claim 12, wherein the expandable member has a toroidal shape and comprises an inflatable pliant balloon.

16. A method, comprising:
delivering a basket to an area of interest within a patient, the basket configured to transition between a first retracted configuration within a sheath and a second deployed configuration extending distally of the sheath, the basket including a longitudinally extending member coupled to a proximal end of the basket, and a closure member extending along the longitudinally extending member and circumferentially about a distal end of the basket, wherein manipulation of the closure member is configured to transition the distal end of the basket between a first closed position and a second open position;
moving the basket to the deployed configuration;
capturing an object of interest within the basket; and
extending the closure member relative to the longitudinally extending member to open the distal end of the basket.

17. The method of claim 16, further comprising:
releasing the object of interest.

18. The method of claim 16, wherein the closure member includes a first portion extending within the longitudinally extending member towards the distal end of the basket, a second portion encircling the distal end of the basket, and a third portion extending within the longitudinally extending member towards the proximal end of the basket.

19. The method of claim 16, further comprising:
retracting the closure member relative to the longitudinally extending member to close the distal end of the basket.

20. The method of claim 16, further comprising:
moving the basket relative to the longitudinally extending member.
